(19) United States
(12) Patent Application Publication
(10) Pub. No.: US 2004/0186668 A1
(43) Pub. Date: Sep. 23, 2004

(54) Library design system and method

(76) Inventors: Valerie Jane Gillet, Sheffield (GB); Darren Victor Steven Green, Stevenage (GB); Peter John Fleming, Sheffield (GB); Peter Willett, Sheffield (GB)

Correspondence Address: FISH & RICHARDSON PC, 225 FRANKLIN ST, BOSTON, MA, 02110, US

(21) Appl. No.: 10/466,501

(22) PCT Filed: Dec. 3, 2001
(86) PCT No.: PCT/GB01/05347

(30) Foreign Application Priority Data
Dec. 1, 2000 (GB) 00293613

(51) Int. Cl. G06F 19/00; G01N 33/48; G01N 33/50

(52) U.S. Cl. 702/19

(57) ABSTRACT

The present invention relates to the design of libraries, such as combinatorial libraries, which may be used in the discovery of novel potentially useful compounds. The invention operates on a population of libraries that is refined iteratively. The refinement involves the following steps: calculating the relative dominance of the libraries in the population; selecting libraries for modification according to dominance; modifying the selected libraries using genetic operators; and inserting the modified libraries back in the population. The refinement steps are repeated until adequate convergence is deemed to have occurred or for a specified number of iterations. The Pareto optimal set of libraries in the final population is output for further processing such as storage or manufacture.

### FIELD OF THE INVENTION

The present invention relates to library design and a system and method therefor.

### BACKGROUND OF THE INVENTION

“Background theory of molecular diversity”, Gillet V J In: Dean P M, Lewis R A, EDS, “Molecular diversity in drug design”, Dordrecht: Kluwer 1999: 43-65 discloses computational methods for the design of combinatorial libraries prior to drug synthesis. The focus of the prior art in combinatorial library design was initially diversity and was founded upon the assumption that libraries, which have broad coverage of chemistry space, will increase the chance of finding new potentially useful compounds. It will be appreciated, however, that there exists practical limits on the sizes of combinatorial libraries which, in turn, leads to a practical chemistry space that is smaller than the maximum theoretical chemistry space. It has in recent times become evident that diversity alone is insufficient to focus research into new compounds since in some regions of a chemistry space there are molecules with properties that make them unlikely drug candidates. Therefore, while diversity is still an important criterion, it is now recognised that other factors should also be taken into account. For example, the physicochemical properties of the molecules that determine effects such as ADME are important as well as other factors such as cost and availability of reactants.

There is a growing interest in the design of focused libraries. Focused libraries are constrained to occupy restricted regions of chemistry space with the boundaries being defined by what is known about the biological target of interest. For example, if a compound active against the target is known, the library could be constrained to contain molecules that are similar to the known that compound. In focused library design it is also desirable to optimise multiple properties since in addition to matching constraints related to the target molecule, other criteria are often required during lead optimisation, for example, bioavailability and cost of goods.

The prior art also comprises a number of methods for designing combinatorial libraries based on a number of properties. For example, these methods can be divided into reactant-based designs and product-based designs. In reactant-based designs, optimised subsets of reactants are selected on the assumption that when reactants from different pools are combined combinatorially an optimised set of products results.

The product-based approaches are typically implemented via an optimisation techniques such as a genetic algorithm see, for example, Gillet V J, Willet P Bradshaw J, Green D V S, “Selecting combinatorial libraries to optimise diversity and physical properties”, J Chem Inf Comput Sci 1999, 39: 169-177 or simulated annealing as disclosed in, for example, Zheng W, Hung S T, Saunders J T, Seibel C L, PICCALO: tool for combinatorial library design via multicriterion optimisation, In: Altman R B, Dunker A K, Hunter L, Lauderdale K, Klein T E, eds. Pacific Symposium on Biocomputing 2000, Singapore: World Scientific, 2000: 588-599 and Good A C, Lewis R A, “New Methodology for Profiling Combinatorial Libraries and Screened Sets: Cleaning up the Design Process with HARPick”, J Med Chem 1997; 40: 3926-3963.

In the well known SELECT program, combinatorial subsets are selected from a fully enumerated virtual library using a standard genetic algorithm such as is shown in the flowchart 100 of FIG. 1 and described hereafter. SELECT uses as an input a virtual library together with molecular descriptors that have been calculated for each molecule within the library.

The library can consist of any number of components or reactant pools. Initially, SELECT was developed to optimise a single objective; namely the diversity of the combinatorial subset using a distance based diversity index.

Each chromosome of the genetic algorithm represents a combinatorial library encoded as reactants selected from each reactant pool.

The genetic algorithm begins with a population of individuals that are initialised with random values at step 102. A chromosome is scored by enumerating the combinatorial subset it represents and measuring its diversity via a fitness function such as, f(n)=diversity.

Conventionally, diversity is measured as the sum-of-pairwise dissimilarities calculated using the cosine coefficient and Daylight fingerprints. However, other diversity indices and other descriptors can also be used. The population is sorted according to fitness.

The genetic algorithm enters an iterative phase where individuals are chosen for reproduction using a roulette wheel parent selection in step 104 and in which reproduction takes place via mutation or crossover via genetic operators in step 106. The newly created individuals are scored and inserted into the population so as to replace the worst individuals and the population is re-sorted in steps 108 to 112. The iterations continue until adequate convergence, measured at step 114, has been achieved. The number of chromosomes selected for reproduction is determined by the replacement rate. A replacement rate of, for example, 10% may be suitable. Within SELECT, sufficient convergence is deemed to have occurred when there has been no change in the fitness of the best individual for a user-specified number of iterations. The parameters of SELECT are configured via an input file. The parameters include characteristics such as, for example, population size, relative rates of crossover versus mutation and the replacement rate. SELECT has been used to demonstrate the benefits of performing product-based library design over reactant-based design.

However, traditional optimisation techniques such as genetic algorithms and simulated annealing have tended to deal with a single optimisation criterion or objective, that is, the maximisation or minimisation of a single measure or quantity.

It will be appreciated, however, that most practical search and optimisation applications should preferably be characterised by the existence of a plurality of fitness measures against which final search results can be judged. For example, as already described, in a library design context, such fitness measures could typically include diversity, some measure of drug-likeness and cost.

However, optimal performance in one objective often implies an unacceptably low performance in at least one of the other objectives. For example, libraries designed using diversity alone as a measure of fitness have a tendency to contain molecules that are not suitable for use as drugs such as, for example, molecules with high molecular weights.

Therefore, it can be appreciated that there is a need to compromise and that the search for solutions must offer acceptable performance in all objectives even though any such acceptable performance may be sub-optimal as measured against any of the individual objectives. A known technique for achieving a compromise over a number of objectives is to combine the objectives via a weighted-sum of fitness functions. For example, SELECT has been extended to perform multi-objective optimisation in a product-space so that other properties, such as, for example, the physicochemical property profiles, of the library can be optimised simultaneously with diversity. Such a suitable fitness function may have the form of f(n)=w₁.diversity+w₂.property1+w₃.property2 . . . , where the weights (w₁, w₂, w₃ etc) are user-defined and the properties (property1, property2, etc) can include physicochemical property profiles such as molecular weight profile or other calculable properties such as costs. Typically, each objective is normalised before being combined.

The advantage of combining multiple objectives via a weighted fitness function is that a single compromise solution is produced. However, such an approach bears the following limitations

(a) a definition of the fitness function can be difficult especially with non-commensurable objectives, for example, it is not obvious how diversity should be combined with cost,

(b) the setting of weights is non-intuitive, typically in the SELECT program the objectives are normalised and then weighted equally,

(c) the fitness function effectively determines the regions of the search space that are explored and can result in some regions being unexplored,

(d) the progress of the search or optimisation process is not easy to follow since there are many objectives to monitor simultaneously,

(e) the objectives may be coupled thus implying conflict or competition, which can make it more difficult for the optimisation process to achieve reasonable or acceptable results

(f) a single solution is found which is typically only one of a family of possible solutions that, while having different values of the individual objectives, are equivalent in terms of the overall fitness, and

(g) when the objectives are non-convex, some solutions will not be obtained using this weighted fitness function method.

Referring to the graph 200 of FIG. 2, which shows the results of several runs of SELECT for a common amide library design problem, some of these limitations can be appreciated. The libraries have been optimised on diversity and molecular weight profile simultaneously via the weighted-sum fitness function:

f(n)=w₁(1−D)+w₂ΔMW

where D is diversity, included in the fitness function as 1−D so that the term w₁(1−D) is minimised; ΔMW is the normalised RMSD between the two profiles. In FIG. 2, the y-axis has been reversed so that diversity increases with distance from the origin and the aim is to find a solution that is as close to the origin as possible on both axes. The triangles show the results found when both weights, w1 and w2, are unity. It can be appreciated that these points form a first cluster 202 in the top left-hand corner of the graph favouring relatively low (good) values of molecular weight with relatively poor values for diversity. Increasing the relative importance of diversity by adjusting the weights to w1=2 and w2=0.5 results in a second cluster 204 of solutions with improved diversity but at the expense of higher values of molecular weight. The second cluster is illustrated using circles. A third cluster 206, illustrated using diamonds, shows the results obtained for w1=10 and w2=1.0. It can be seen that the distribution has been shifted further in favour of diversity at the expense of the molecular weight profile of the library. Each of the solutions represents a different compromise between the two objectives and in terms of overall fitness. All of these solutions appear to be equally valid. It can be appreciated from the above that full coverage of the search space using a weighted-sum fitness function requires many runs of SELECT to be performed using different weights to find an acceptable solution. This is clearly a time consuming, slow and computationally intensive constraint.

It is an object of the present invention at least to mitigate some of the problems of the prior art.

### SUMMARY OF THE INVENTION

Accordingly, a first aspect of the present invention provides a method for designing a set of libraries using a population of libraries, the method comprising performing, at least once, the steps of:

selecting at least a plurality of the libraries from the population of libraries;

applying genetic operators to selected, ranked, libraries to produce modified libraries;

calculating each of a plurality of objectives for each of the modified libraries;

calculating an associated dominance indication of each of the modified libraries;

ranking the modified libraries according to associated dominance indications;

incorporating the modified libraries into the population of libraries; and

forming the set libraries comprising selecting at least one library from the population of libraries.

Advantageously, applying such a multi-objective optimisation technique to the problem of library design results in a family of alternative solutions that are all considered to be equivalent. Furthermore, multiple solutions arise in situations, which include, for example, the case of two competing objectives. Still further, as the number of objectives increases, it will be appreciated that the problem of finding a satisfactory compromise solution becomes increasingly complex. However, since the embodiments of the present invention operate with a population of individuals, the embodiments are well suited to search for multiple solutions in parallel and are applicable readily to multi-objective search and optimisation of combinatorial library design.

Preferably, embodiments provide a method in which the set of libraries is at least one of a set of combinatorial libraries or near combinatorial libraries.

Embodiments preferably provide a method in which the population of libraries is a population of combinatorial libraries or near combinatorial libraries.

Still further, embodiments provide a method in which the modified libraries are at least one of modified combinatorial libraries or modified near combinatorial libraries.

In preferred embodiments, there is provided a method in which the step of selecting at least one library from the population of libraries comprises the step of selecting at least one combinatorial and/or near combinatorial library from the population of libraries.

Preferred embodiments provide a method in which the step of forming the set of libraries comprises the step of forming a Pareto set of libraries.

Preferably, the Pareto set is a Pareto optimal set.

Preferred embodiments provide a method in which the plurality of objectives are specified via at least an n-dimensional vector function (f) of a population library (x) and at least two n-dimensional objective vectors (u=f(xᵤ) and v-f(xᵥ)).

Still further, embodiments preferably provide a method in which the step of ranking the modified libraries comprises the step of determining an order of preference of the modified libraries.

Preferred embodiments provide a method in which the step of determining an order of preference of the modified libraries comprises determining that at least one of the objective vectors (u=[u₁, . . . , uₚ]) for a first modified library is preferable to the at least one of the objective vectors (v=[v₁, . . . , vₚ) for a second modified library given a preference vector (g=[g₁, . . . , gₚ])

$$( u {≺}_{g} v )$$

if and only if

p=1 [symbol] (uₚ′ₚ<vₚ′)=>{(uₚ′=vₚ′)

\hat{ }[(vₚ*not≦gₚ*)=>(uₚ*ₚ<vₚ*)]}

and

p>1 [symbol] (uₚ′ₚ<vₚ′)=>{(uₚ′=vₚ′)

where u_{i, . . . ,p-1}32 [u_{i, . . . ,} uₚ₋₁]and similarly for v and g; where the first kᵢ components of vectors uᵢ,vᵢ, and gᵢ are represented as uᵢ *, vᵢ*, and gᵢ*, respectively; the last nᵢ-kᵢ component of the same vectors are denoted uᵢ′, vᵢ′, and gᵢ′, also respectively; and the * and ′ indicate the components in which u either does or does not meet the goals.

A preferred embodiment provides a method in which the step of calculating the associated dominance indication of each of the modified libraries comprises determining whether at least a first objective vector (u=(u₁, . . . , uₙ)) for a first modified library has Pareto dominance over a second objective vector (v=(v₁, . . . , vₙ)) for a second modified library if and only if the u is partially less than v (uₚ<v) such that ∀iε{1, . . . ,n},uᵢ≦vᵢ=>∃iε{1, . . . , n}:uᵢ<vᵢ.

Preferably, embodiments provide a method in which the step of ranking the modified library comprises the steps of evaluating the preference of each modified library and ranking the modified library according to respective preferences.

Preferred embodiments provide a method in which the step of forming the set of libraries comprises the step of selecting the ranked modified libraries that are Pareto-optimal where a first library (xᵤ) of the population for a first objective vector is said to be Pareto-optimal if and only if there is no other library of the population for a second objective vector (xᵥ) for which the second objective vector, v=f(xᵤ)=(v₁, . . . , vₙ) dominates the first objective vector u=f(xᵤ)=(u₁, . . . , uₙ).

A further aspect of the present invention provides a method for designing a set of combinatorial libraries using a population of combinatorial libraries, the method comprising performing, at least once, the steps of:

selecting at least a plurality of the combinatorial libraries from the population of combinatorial libraries;

applying genetic operators to selected, ranked, combinatorial libraries to produce modified combinatorial libraries;

calculating each of a plurality of objectives for each of the modified combinatorial libraries;

calculating an associated dominance indication of each of the modified combinatorial libraries;

ranking the modified combinatorial libraries according to associated dominance indications;

incorporating the modified combinatorial libraries into the population of combinatorial libraries; and

forming the set combinatorial libraries comprising selecting at least one combinatorial library from the population of combinatorial libraries.

Preferably, embodiments provide a method in which the step of forming the set of combinatorial libraries comprises the step of forming a Pareto set of combinatorial libraries.

Preferably, a method is provided in which the Pareto set is a Pareto optimal set.

Embodiments provide a method in which the plurality of objectives are specified via at least an n-dimensional vector function (f) of a population library (x) and at least two n-dimensional objective vectors (u=f(xᵤ) and v=f (xᵥ)).

Preferred embodiments provide a method in which the step of ranking the modified combinatorial libraries comprises the step of determining an order of preference of the modified combinatorial libraries.

Preferably, embodiments provide a method in which the step of determining an order of preference of the modified combinatorial libraries comprises determining that at least one of the objective vectors (u=[u₁, . . . , uₙ]) for a first modified combinatorial library is preferable to the at least one of the objective vectors (v=[v₁, . . . , vₚ]) for a second modified combinatorial library given a preference vector (g=[g₁, . . . , gₚ])

$$( u {≺}_{g} v )$$

if and only if

p=1=>(uₚ′ₚ<vₚ′)=>{(uₚ′=vₚ′)

\hat{ }[(vₚ*not≦gₚ*)=>(uₚ*ₚ<vₚ*)]}

and

p>1=>(uₚ′ₚ<vₚ′)=>{(uₚ′=vₚ′)

where u_{i, . . . ,p-1}=[uᵢ, . . . , uₚ₋₁]and similarly for v and g; where the first kᵢ components of vectors uᵢ,vᵢ, and gᵢ are represented as uᵢ*, vᵢ*, and gᵢ*, respectively; the last nᵢ-kᵢ component of the same vectors are denoted uᵢ′, vᵢ′, and gᵢ′, also respectively; and the * and ′ indicate the components in which u either does or does not meet the goals.

Preferred embodiments provide a method in which the step of calculating the associated dominance indication of each of the modified combinatorial libraries comprises determining whether at least a first objective vector (u=(u₁, . . . , uₙ)) for a first modified combinatorial library has Pareto dominance over a second objective vector (v=(v₁, . . . , vₙ)) for a second modified combinatorial library if and only if the u is partially less than v (uₚ<v) such that ∀iε{1, . . . ,n}uᵢ≦vᵢ=>∃iε{1, . . . , n}:uᵢ<vᵢ.

Preferred embodiments provide a method as claimed in which the step of ranking the modified combinatorial library comprises the steps of evaluating the preference of each modified combinatorial library and ranking the modified combinatorial library according to respective preferences.

Preferably, there is provided a method in which the step of forming the set of combinatorial libraries comprises the step of selecting the ranked modified combinatorial libraries that are Pareto-optimal where a first combinatorial library (xᵤ) of the population for a first objective vector is said to be Pareto-optimal if and only if there is no other combinatorial library of the population for a second objective vector (xᵥ) for which the second objective vector, v=f(xᵥ) =(v₁, . . . ,vₙ) dominates the first objective vector u=f(xᵤ)=(u₁, . . . , uₙ).

Preferred embodiments provide a method substantially as described herein with reference to and/or as illustrated in the accompanying drawings.

A still further aspect of the present invention provides a system for designing a set of combinatorial libraries using a population of combinatorial libraries, the system means for invoking, at least once: means for selecting at least a plurality of the combinatorial libraries from the population of combinatorial libraries;

means for applying genetic operators to selected, ranked, combinatorial libraries to produce modified combinatorial libraries;

means for calculating each of a plurality of objectives for each of the modified combinatorial libraries;

means for calculating an associated dominance indication of each of the modified combinatorial libraries;

means for ranking the modified combinatorial libraries according to associated dominance indications;

means for incorporating the modified combinatorial libraries into the population of combinatorial libraries; and means for forming the set combinatorial libraries comprising selecting at least one combinatorial library from the population of combinatorial libraries.

Preferably, embodiments are arranged to implement the system equivalents of the above-described methods and the methods described herein.

Preferably, embodiments provide a combinatorial library design computer program element for implementing a method or system.

Preferred embodiments provide a computer program product comprising a computer readable storage medium having stored thereon a computer program element.

Preferred embodiments provide a method of manufacturing a combinatorial library or element thereof comprising the steps of designing the combinatorial library or element using a method, system, computer program element or computer program product as claimed in any preceding claim; and materially producing the designed combinatorial library or element thereof.

### BRIEF DESCRIPTION OF THE DRAWINGS

Embodiments of the present invention will now be described, by way of example only, with reference to the accompanying drawings in which:

FIG. 1 illustrates a flow chart for implementing the SELECT processing steps according to the prior art;

FIG. 2 shows combinatorial libraries for different weightings of two objectives; namely diversity and molecular weight profile according to the prior art;

FIG. 3 shows a flow chart for implementing an embodiment of the present invention;

FIG. 4 illustrates libraries that can be used with the embodiments of the present invention;

FIGS. 5a and 5b illustrate the progress of a search according to an embodiment;

FIG. 6 illustrates a distribution of Pareto solutions for 10 runs of an embodiment of the present invention;

FIG. 7 depicts Pareto frontiers for 10 runs of an embodiment with convergence for selecting 30×30 combinatorial subsets from a 10K amide library;

FIG. 8 depicts results of an embodiment using niche induction;

FIG. 9 shows the distribution of overlap in an embodiment using clustering;

FIG. 10 shows a parallel co-ordinates graph representation of the results of a two-objective problem illustrated in FIGS. 5a and 5b;

FIG. 11 shows a plurality of parallel co-ordinates graph representations of the progress of a search according to an embodiment for a multi-objective optimisation of a 30×30 amide library;

FIG. 12 shows a parallel co-ordinates graph representation of Pareto frontiers at initialisation and after 5000 iterations of an embodiment arranged to select 15×30 combinatorial subsets of a 2-aminothiazole library; and

FIG. 13 shows an embodiment of a two-objective problem in focused library design where 15×30 combinatorial subsets are selected from a 2-aminothiazole library optimised on similarity to a target molecule and cost.

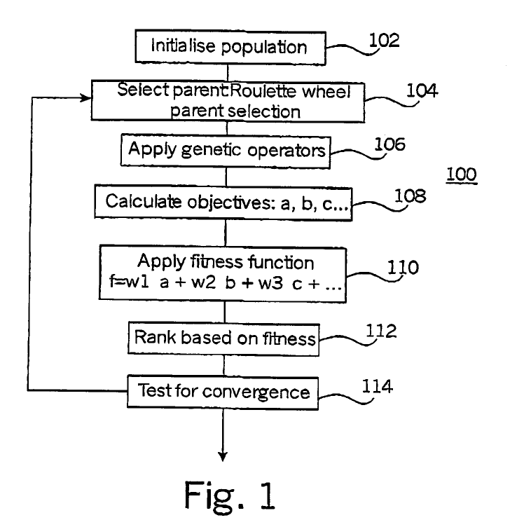

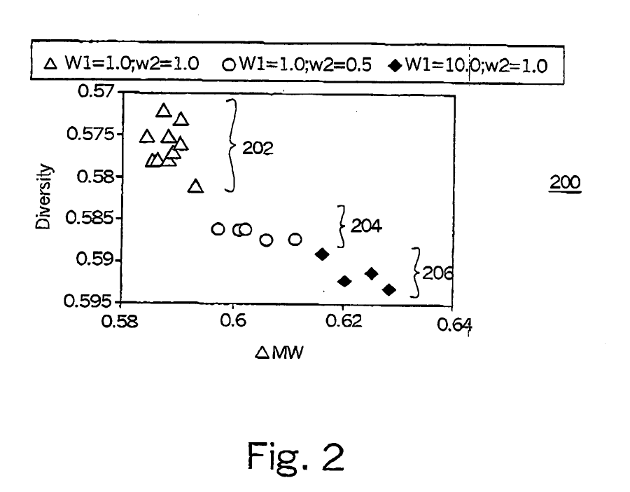

### DESCRIPTION OF THE PREFERRED EMBODIMENTS

The embodiments of the present invention utilise a population-based search method (for example, an evolutionary algorithm) in which the multiple objectives are handled independently. An embodiment produces a hyper-surface within a population search space that represents a continuum of solutions where all solutions on that hyper-surface are equivalent (in contrast to the single solution produced by SELECT). The hyper-surface represents a compromise between the objectives optimised by the embodiment. The embodiment can produce a plurality of types of solution which are known as trade-off, non-dominated, non-inferior, superior or Pareto solutions. The embodiments of the present invention preferably operate to produce a set of non-dominated solutions rather than a single solution as is the case in SELECT.

Before explaining the nature of the embodiments of the present invention, it is necessary to define several terms and operators used in the embodiments. Consider an n-dimensional vector function f of some decision variable x and two n-dimensional objective vectors u=f(xᵤ) and v=f(xᵥ), where xᵤ and xᵥ are particular values of x. Consider also the n-dimensional preference vector

$$g = [ {g}_{1} , … , {g}_{p} ] \\ = [ ( {g}_{1 , 1} … , {g}_{1 , n1} ) , … , ( {g}_{p , 1} , … , {g}_{p , np} ) ]$$

where p is a positive integer (see below), nᵢε{0, . . . , n} for i=1, . . . , p, and

$${\sum }_{i = 1}^{p} {n}_{i} = n .$$

Similarly, u may be written as

$$u = [ {u}_{1} , … , {u}_{p} ] \\ = [ ( {u}_{1 , 1} , … , {u}_{1 , n1} ) , … , ( {u}_{p , 1} , … , {u}_{p} {,}_{np} ) ]$$

and the same for v and f

The subvectors gᵢ of the preference vector g, where i=1, . . . , p, associate priorities i and goals g_{i,j}, where jᵢ=1, . . . , nᵢ, to the corresponding objective functions ƒ_{i,j1}, components of fᵢ. This assumes a convenient permutation of the components of f, without loss of generality. Greater values of i, up to and including p, indicate higher priorities.

Generally, each subvector uᵢ will be such that a number kᵢε{0, . . . , nᵢ} of its components meet their goals while the remaining do not. Also without loss of generality, u, is such that, for i=1, . . . , p, one can write

∃kᵢε{0, . . . , nᵢ}|∀lε{1, . . . ,kᵢ},

∀mε{kᵢ+1, . . . , nᵢ},(u_{i,l}≦g_{i,l})\hat{ }(u_{i,m}>g_{i,m}).

For simplicity, the first kᵢ components of vectors uᵢ,vᵢ, and gᵢ will be represented as uᵢ*, vᵢ*, and gᵢ*, respectively. The last nᵢ-kᵢcomponent of the same vectors will be denoted uᵢ′, vᵢ′, and gᵢ′, also respectively. The * and ′ indicate the components in which u either does or does not meet the goals.

Definition (Preferability): Vector u=[uᵢ, . . . , uₚ] is preferable to v=[vᵢ, . . . , vₚ] given a preference vector

$$g = {g}_{i} , … , {g}_{p} ] ( u {≺}_{g} v ) iff$$

p=1=>(u_{p′}ₚ<vₚ′)=>{(uₚ′=vₚ′)

\hat{ }[(vₚ*not≦gₚ*)=>(uₚ*ₚ<vₚ*)]}

and

p>1=>(uₚ′ₚ<vₚ′)=>{(uₚ′=vₚ′)

where u_{i, . . . ,p-1}=[uᵢ, . . . , uₚ₋₁] and similarly for v and g.

Note: uₚ<V denotes u is partially less than v, i.e.

∀iε{1, . . . , n}, uᵢ≦vᵢ\hat{ }∃iε(1, . . . ,n}:uᵢ<vᵢ.

In simple terms, vectors u and v are compared first in terms of their components with the highest priority, that is, those where i=p, disregarding those in which up meets the corresponding goals uₚ*. In case both vectors meet all goals with this priority, or if they violate some or all of them, but in exactly the same way, the next priority level (p-1) is considered. The process continues until priority 1 is reached and satisfied, in which case the result is decided by comparing the priority 1 components of the two vectors in a Pareto fashion.

Since satisfied high-priority objectives are left out from comparisons, vectors which are equal to each other in all but these components express virtually no trade-off information given the corresponding preferences. The following symmetric relation is defined.

Definition (Equivalence): Vector u=[uᵢ, . . . ,uₚ] is equivalent to v=[v₁, . . . ,vₚ] given a preference vector

$$g = [ {g}_{1} , … , {g}_{p} ] {( u ≡ v )}_{g} iff$$

(u′=v′)\hat{ }(uᵢ*=v₁*)\hat{ }(v*_{2, . . . ,p}≦g*_{2, . . . ,p}).

The concept of preferability can be related to that of inferiority as follows:

Lemma 1: For any two objective vectors u and v, if uₚ<V, then u is either preferable or equivalent to v, given any preference vector g=[g₁, . . . ,gₚ].

Lemma 2: (Transitivity): The preferability relation is transitive, i.e. given any three objective vectors u,v, and w, and a preference vector g=[g₁, . . . ,gₚ]

$$u {≺}_{g} v {≺}_{g} w {⇒}_{g} u {≺}_{g} w .$$

Particular Cases: The decision strategy described above encompasses a number of simpler multi-objective decision strategies which correspond to particular settings of the preference vector.

Pareto (Definition 1): All objectives have equal priority and no goal levels are given g=[g₁]=[(−∞, . . . −∞_].

Lexicographic: Objectives are all assigned different priorities and no goal levels are given. g=[g₁, . . . , gₙ]=[(−∞), . . . ,(−∞)].

Constrained Optimisation: The functional parts of a number n_{c} of inequality constraints are handled as high priority objectives to be minimised until the corresponding constraint parts, the goals, are reached. Objective functions are assigned the lowest priority. g=[g₁,g₂]=[(−∞, . . . , −∞),(g_{2,1}, . . . g_{2,n}_{_{c}})].

Constraint Satisfaction: All constraints are treated as in constrained optimisation, but there is no low priority objective to be optimised. g=[g₂]=[(g_{2,1}, . . . , g_{2,n})].

Goal Programming: Several interpretations of goal programming can be implemented. A simple formulation consists of attempting to meet the goals sequentially, in a similar way to lexicographic optimisation. g=[g₁, . . . , gₙ]=[(g_{1,1}), . . . , (g_{n,1})].

A second formulation attempts to meet all the goals simultaneously, as with constraint satisfaction, but requires solutions to be satisfactory and Pareto optimal. g=[g₁]=[(g_{1,1}, . . . , g_{1,n})].

Population ranking. As opposed to the single objective case, the ranking of a population in the multi-objective case is not unique. In the present embodiment, it is desirable that all preferred combinatorial libraries or individuals are placed higher in rank than those to which they are preferable. For example, consider an individual xᵤ at a generation t with a corresponding objective vector u, and let rᵤ^{(t)}, be the number of individuals in the current population which are preferable to it. The current position of xᵤ in the individuals' rank can be given by rank (xᵤ,t)=rᵤ^{(t)}, which ensures that all preferred individuals in the current population are assigned rank zero.

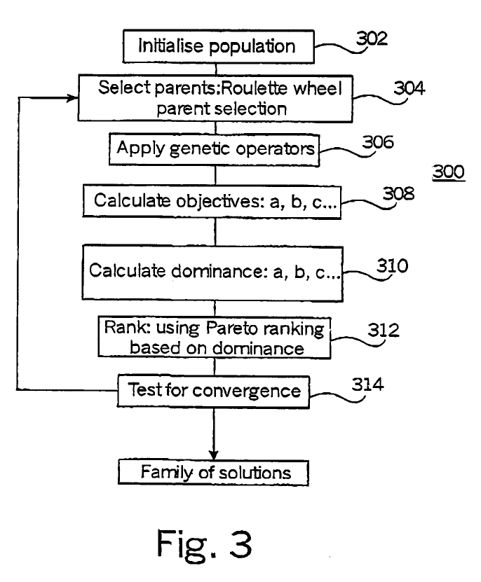

FIG. 3 illustrates a flow chart for an embodiment of the present invention in which a multi-objective genetic algorithm is used as an illustration of a population-based search method. In step 302, the optimisation to be solved is initialised, that is, the population is initialised. The definitions of chromosomes and the reproduction operators used in the embodiment are substantially the same as those used in SELECT.

Referring again to FIG. 3, at step 304, a parent selection technique, such as roulette wheel parent selection, is used to select the combinatorial library or parents from the initialised population based on dominance. It will be appreciated that many chromosomes may have the same rank, for example, all chromosomes on the Pareto frontier have rank of zero. Accordingly, step 304 sorts the population using normalised fitness values as follows

(a) the population is sorted according to a predeterminable rank, such as that described above,

(b) fitness assignments are undertaken by interpolating from the best individual (rank =zero) to the worst individual (rank=max r^{(t)}<N) according to some function, which is usually linear or exponential, and

(c) the fitness assigned to individuals with the same rank is averaged so that all such individuals are sampled at the same rate while keeping the global population fitness constant.

Hence, according to the present embodiment, a parent chromosome is chosen with a probability that is proportional to the normalised fitness value of that chromosome. By way of contrast, in SELECT the fitness value, that is, the weighted-sum over each objective, is used to sort the chromosomes in rank order with the fittest appearing at the top of the list and a parent chromosome is chosen with a probability that is proportional to the ranked position of that chromosome.

A predetermined number of chromosomes are selected in a first pass in step 304. In step 306, as with the SELECT technique, the genetic operators are applied to the selected parent chromosomes to produce modified or mutated chromosomes or modified combinatorial libraries. Step 308 calculates the objectives, that is, the objective vectors, using the mutated chromosomes that were produced by the application of the genetic operators in step 306. Having calculated the objectives, the dominance of the results of calculating the objectives are assessed in step 310 and the chromosomes are ranked based on dominance in step 312. The population is optionally tested for convergence at step 314. If sufficient convergence has occurred or if a user-defined number of iterations have been completed, the processing terminates and the current chromosomes or at least a selection thereof are output as offering Pareto optimal solutions. However, if insufficient convergence has occurred or an insufficient number of iterations have been completed, processing continues, at step 304, to select new parent chromosomes from the population of chromosomes that include both the original chromosomes and the newly derived chromosomes. Preferably, the newly derived chromosomes replace a pre-determinable number of the least suitable chromosomes after ranking.

Examples of the application of the present invention to combinatorial chemical library design will be described hereafter.

### EXAMPLE 1

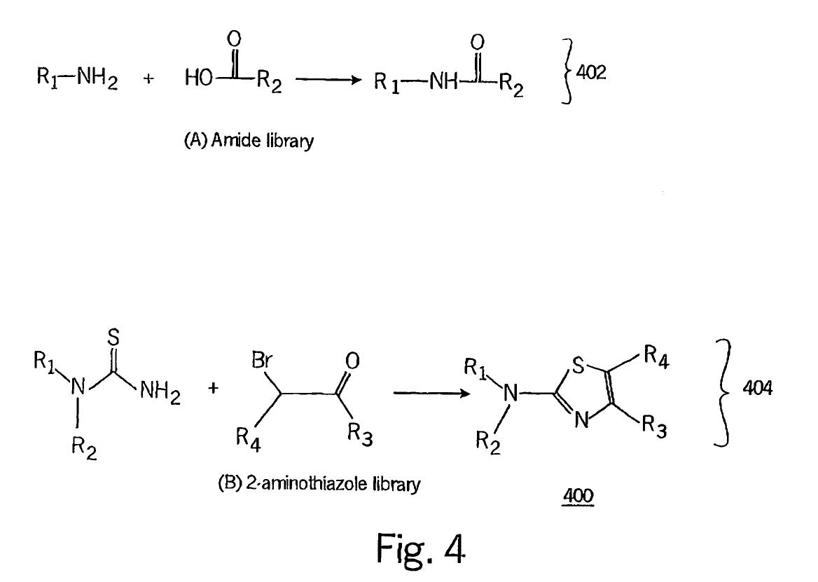

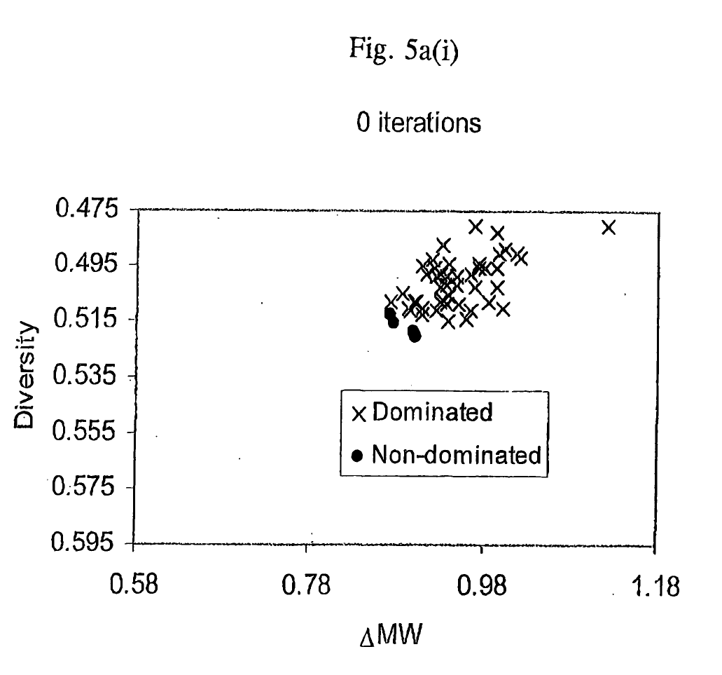

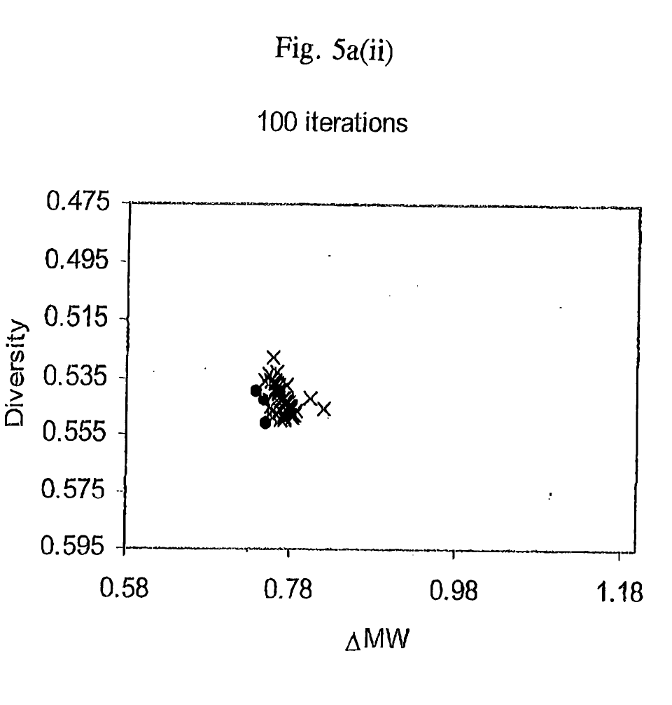

Referring to FIG. 4, there is shown two virtual libraries 400 comprising a two-component amide library 402 and a two component 2-aminothiazole library 404. The amide library 402 represents a virtual library of 10,000 components formed by the coupling of 100 amines and 100 carboxylic acids, extracted at random from the SPRESI database as is well known within the art.

The 2-aminothiazole virtual library 404 comprises 12,850 virtual products generated by reacting 74 α-bromoketones with 170 thioureas. In this case, the reactants for each pool were obtained from the available chemicals directory (ACD), as is known in the art, and filtered using ADEPT software, as is also known within the art, to remove reactants having molecular weights of greater than 300 and more than 8 rotatable bonds.

Furthermore, in the present example, a series of reactants that contained undesirable substructural fragments were removed by way of a series of substructure searches.

In the initialisation step 302 of FIG. 3, each virtual library was enumerated and various properties were calculated for the product molecules comprised in each library [1024 bit Daylight fingerprints, molecular weight (MW), number of rotatable bonds (RB), number of hydrogen bond donors (HBD), and number of hydrogen bond acceptors (HBA)].

Unless otherwise stated, diversity was calculated as the sum of pairwise dissimilarities using the cosine coefficient as is known within the art. In the examples presented here the virtual libraries are enumerated and the descriptors are calculated during initialisation. However the present invention can also be applied when libraries are enumerated and descriptors are calculated on-the-fly.

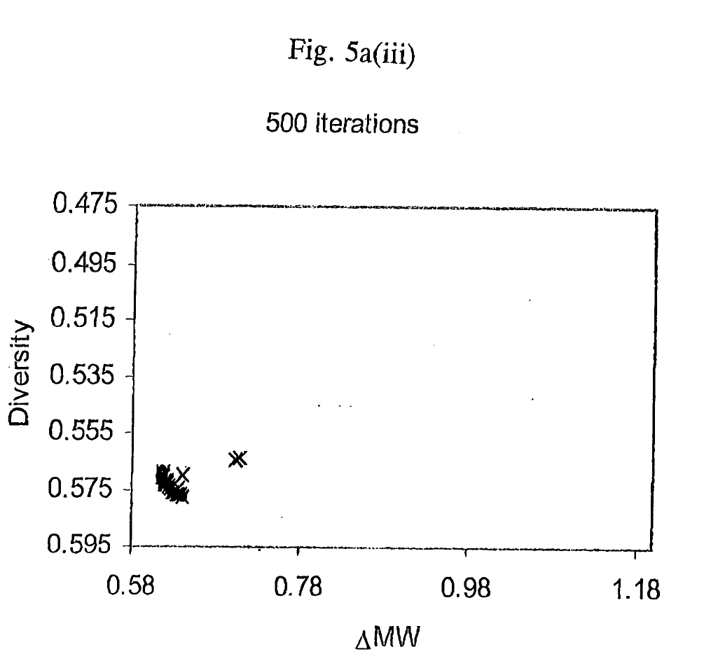

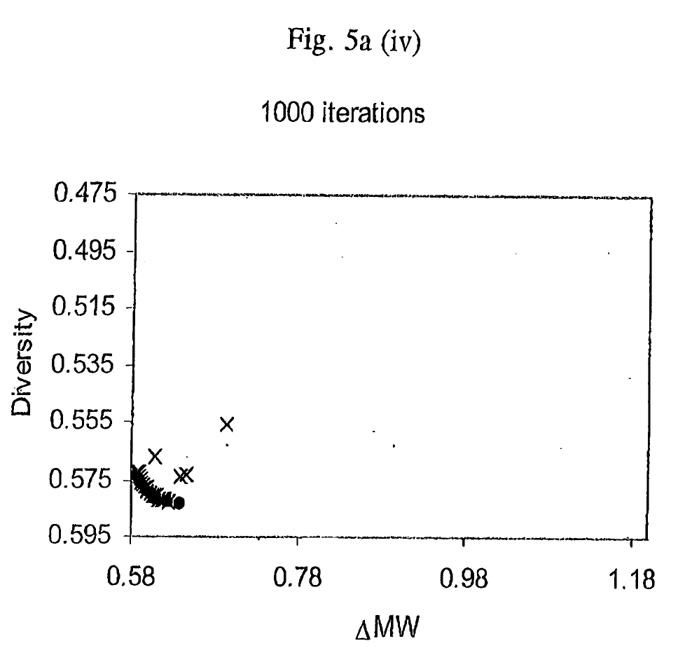

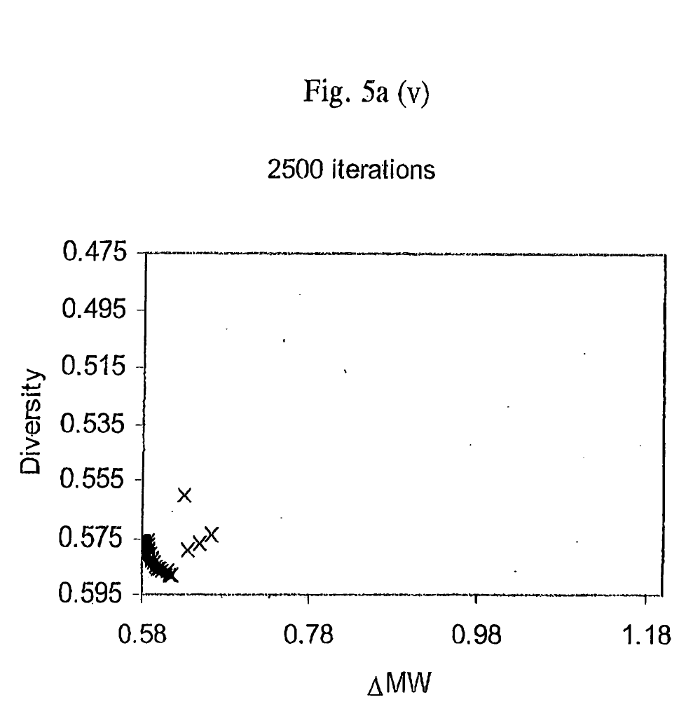

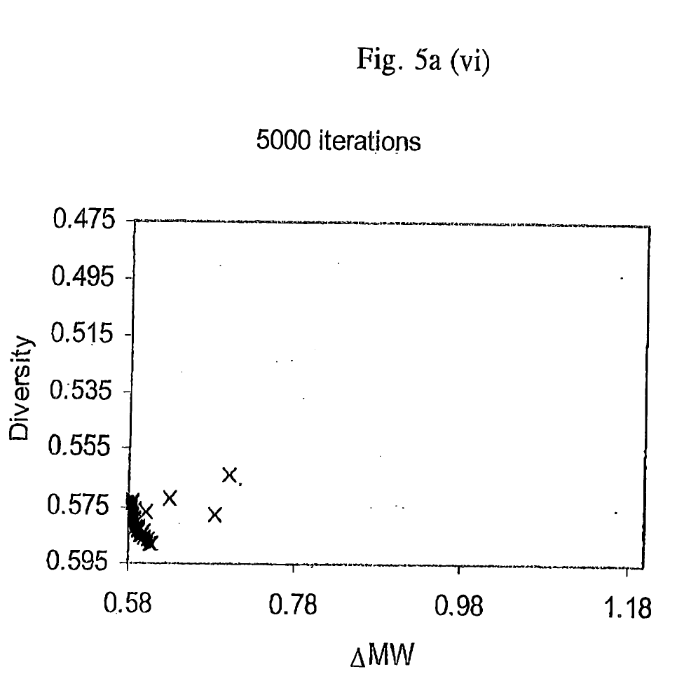

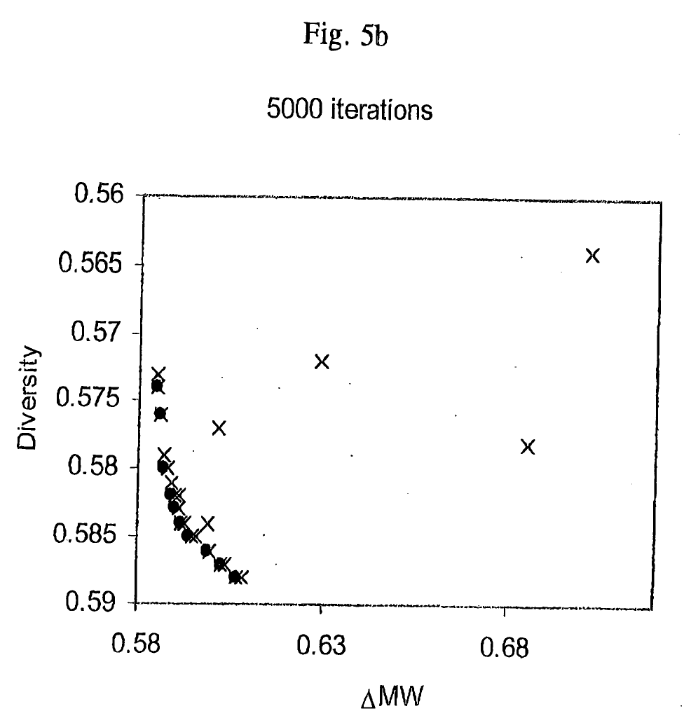

The aim of the first example is to select 30×30 combinatorial subsets from the 10,000 amide virtual library using two objectives; namely, diversity and molecular weight profile. The aim was to maximise diversity while minimising the RMSD between the molecular weight profile of the library and the molecular weight profile found in WDI. The embodiment was run for 5000 iterations with a population size of 50. The progress of the search is shown in FIGS. 5a and 5b. The 5,000^{th} iteration of FIG. 5a is shown enlarged in FIG. 5b. Again, it will be appreciated that the y-axis is arranged so that diversity increases as the origin is approached and the direction of improvement for both objectives is towards the bottom left-hand corner of the graph.

In each of the graphs shown in FIGS. 5a and 5b, the Pareto frontier, that is, the set of non-dominated individuals in a current population, is represented by circles. It can be appreciated from the graphs shown in FIG. 5a, that is, the graphs for iterations 0, 100, 500, 1000, 2500 and 5000, that there is an advancement of the Pareto surface 502, 504, 506, 508, 510 and 512.

It can be appreciated that beyond the first 2,000 iterations there is little improvement in the Pareto set over the subsequent 3,000 generations. However, the percentage of solutions that are non-dominated increases from 4 in the initial population to 17 in the final population shown in the Pareto set 512 of FIG. 5b. The result of the search is family of solutions all of which can be seen as equivalent.

Optionally, once presented with this information, a user can then browse through the solutions and choose acceptable solutions based on the objectives used in the search and optionally, taking into account other criteria such as, for example, the availability of reactants. This is in contrast to the use of the SELECT technique where the search results in a single solution that may not be acceptable.

Alternatively, the final selection may be automated. The automation may be based on the Pareto set meeting a predetermined criterion or predetermined criteria.

### EXAMPLE 2

The next example was designed to compare the performance of the present embodiment with that of SELECT for the above library. SELECT was run 30 times with a population size of 50 and with the two objectives normalised and equally weighted. The convergence criterion was set so that the run was terminated when no change (within a pre-determinable tolerance) was seen in the fitness function over 5 runs, each of 50 iterations. A 10% replacement strategy was used where, in each iteration, at least 5 individuals were modified by applying the genetic operators of mutation and crossover. The embodiment of the present invention using the amide library described above, was repeated for 10 runs and the family of non-dominated solutions was determined at the end of each run. Finally, the SELECT technique was arranged to optimise each objective separately to find optimised values for each objective independently. The values found over 10 runs were an average of 0.592, with standard deviation of 0.002, for diversity and an average of 0.585 for ΔMW with a standard deviation of 0.005.

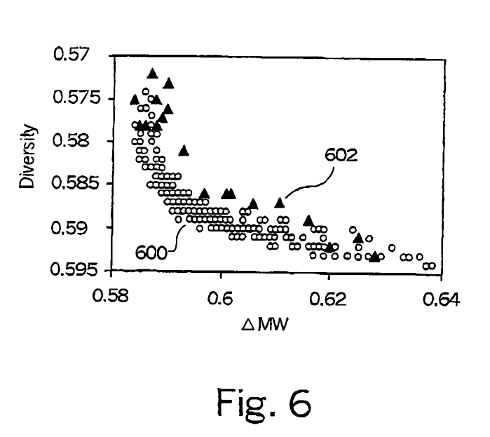

It can be appreciated from FIG. 6 that the final non-dominated solutions found in the 10 runs of the present embodiment, which are shown by circles 600, are preferred over the single best solutions found for the SELECT runs, which are shown as triangles 602. The even-spread of points arising from the embodiment shows the Pareto frontier to have been mapped efficiently. The runs according to the embodiment also include solutions at the extremes, that is, solutions that are found when the objectives are optimised independently. Some variation is seen in the results obtained in the embodiment. However, even the worst family of solutions found contains individuals that are preferable to many of the SELECT solutions. Each triangle 602 represents a single solution produced by a different run of SELECT and the SELECT solutions typically lie somewhere on the Pareto frontier of a single run of the present invention. In effect, the SELECT solutions are single solutions in contrast to the family of solutions produced by the embodiments of the present invention. It will be appreciated that a disadvantage of the SELECT technique is that each time a run is performed a different solution may be obtained. There is no guarantee, by multiple runs, that the complete Pareto frontier being mapped. It has been found that a single run of an embodiment of the present invention maps more of the Pareto frontier than can be achieved over many runs of SELECT.

### EXAMPLE 3

Referring again to FIG. 3, it can be seen in step 314 that a convergence test may be performed. Again, by way of comparison with SELECT, the convergence criterion of SELECT is used to terminate the search when no change was seen in the fitness function of the best individual solution over, for example, 250 iterations (measured at 50 iteration intervals). The aim of the embodiment of the present invention is to identify a family of non-dominated solutions, all of which are equally valid but which have different values of the objectives. Therefore, there is no longer a single fitness value assigned to a potential solution. Thus, the convergence criterion used in SELECT is inappropriate for the present invention.

The aim of example 3 was to investigate the effect of a convergence criterion that has been implemented in embodiments of the present invention. The first criterion attempts to determine the progress of the Pareto frontier, as a whole, or at least a part thereof, rather than the progress of a single best solution. Once an initial population has been created, a copy of the non-dominated set of that initial population is maintained. The search proceeds for a predeterminable number of iterations, for example, 50, after which the current non-dominated set is compared with the previously stored non-dominated set. If none of the chromosomes of the previous non-dominated set are dominated by the current non-dominated set, the Pareto front is deemed to be unchanged over the 50 iterations and the previous non-dominated set is replaced by the current non-dominated set to allow the search to continue for a further cycle of 50 iterations. However, if the Pareto front is unchanged over 250 iterations, the search is terminated.

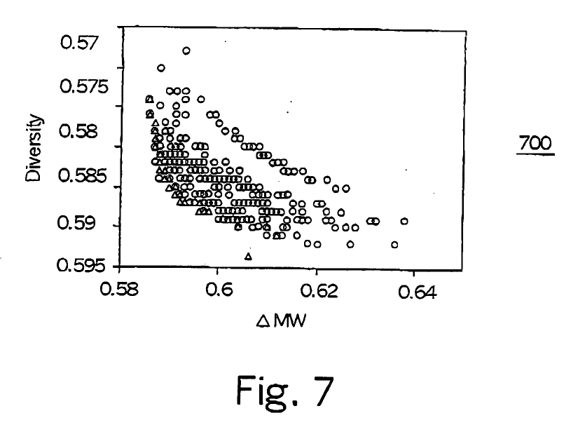

Referring to FIG. 7 there is shown a graph 700 that illustrates the distribution of Pareto frontiers over 10 runs of an embodiment of the present invention with the above convergence criterion. It can be appreciated that the distribution is similar to the distribution shown in FIG. 6 where a convergence criterion was not applied. It can be seen from FIG. 7 that there appears to be some loss of coverage of the extreme values and that the spread of frontiers is broader, which provides an indication of some loss of robustness. Despite the small loss of coverage, the use of such convergence criterion can be advantageous since the results are achieved for a significantly reduced number of cycles.

By way of comparison, the mean number of iterations to convergence for the embodiment is 1715 (and the standard deviation 525), compared to the 5000 iterations shown in FIG. 6, and a mean of 1245 (standard deviation 291) iterations for the SELECT runs. It should be noted that while the numbers of iterations to convergence, as between the embodiments of the present invention and SELECT, are roughly similar, a single run of an embodiment of the present invention produces an entire family of equivalent solutions in contrast to the single solution produced by a single run of SELECT.

### EXAMPLE 4

The multi-objective genetic algorithm, which is used to illustrate the population based approach, is prone to genetic drift or speciation, which manifests itself as a tendency to produce solutions in search space where there are clusters of closely matched solutions to the detriment of the quality of the search in other search spaces. Accordingly, an embodiment provides a method in which the effective speciation is reduced by using a niche induction technique. The density of solutions within a given type of volume of either a decision or objective variable space is restricted. In an embodiment, the objective space was used to attempt to spread the distribution of solutions over a Pareto frontier. After each iteration, the Pareto frontier is identified and each solution on the frontier is compared with all others to establish relative proximity of the solutions within the objective variable space. Preferably, this is implemented as an order dependent process where the first solution encountered is deemed to be positioned at the centre of a hyper-volume or niche. If the difference in the objectives of the next solution and the objectives of any solutions that already form centres of respective niches is within a given threshold, for all objectives, a rank of the current solution forms the centre of a new niche. Such a threshold is known as a niche radius. Preferably, this process is repeated for all solutions on the Pareto frontier. In a preferred embodiment, the niche radius can be varied throughout a run and is given as a percentage of the range of values that exist for each objective on a current Pareto frontier.

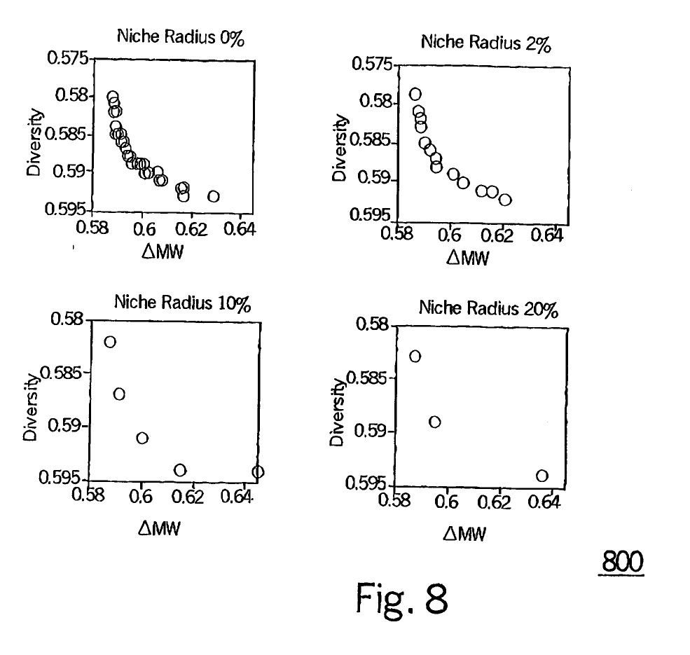

Referring to FIG. 8, there is shown a plurality of graphs 800 which illustrate the relationship between diversity, molecular weight and niche radius. It can be appreciated that there is a loss of resolution as the niche radius is increased.

In an embodiment, niche induction can be applied after each iteration even in the absence of speciation to increase the efficiency of the search since there will be fewer solutions to explore on a corresponding Pareto frontier.

Furthermore, an embodiment applies niche induction once the iterations have been completed to choose a subset of solutions that are distributed across the Pareto frontier.

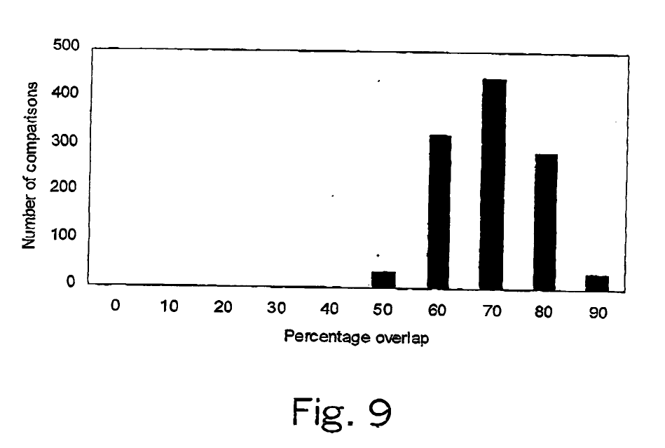

In an alternative embodiment, the above described niche induction can be applied to increase the efficiency and effectiveness of the search. However, in still further alternative embodiments, the above niche induction can be used as a means of clustering a final Pareto set according to the spread of solutions within an object of the space. Alternatively, the solutions can be clustered according to their similarity in terms of the product molecules or the reactants contained within the libraries. FIG. 9 illustrates the results of an embodiment of such clustering for the amide library above to select 30×30 subsets from the 100×100 virtual library. An embodiment of the present invention was run to generate a final Pareto set comprising 48 solution libraries. A pairwise overlap matrix was constructed for the 48 libraries, where the overlap between any two libraries was calculated as the number of product molecules common to the libraries divided by the library size. The distribution of overlap values is as shown in FIG. 9. It can be appreciated that it is possible to group the libraries into clusters according to their overlap in terms of the product molecules contained therein. The selection of a library from a cluster could, in an embodiment, be performed on the basis of the values of the objectives. An embodiment may implement niche induction during the search process itself based on library comparisons in terms of product molecules rather than based on a comparison of objective space as described above.

### EXAMPLE 5

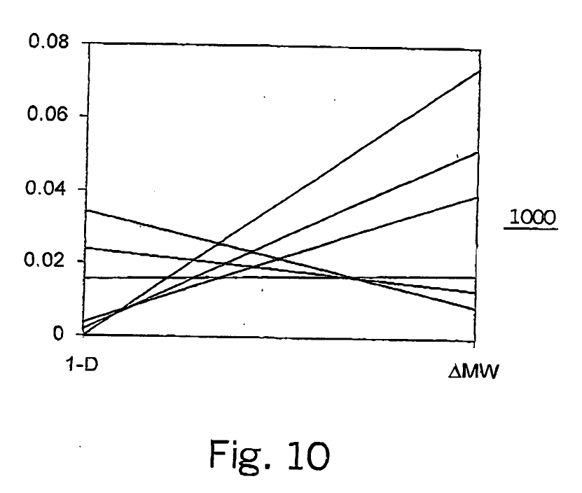

Although the above embodiments have been described with reference to the library design based on two objectives, the present invention is not limited thereto. Embodiments can be realised in which the number of objectives is greater than two. For example, the same amide library could be used with the following five objectives, that is: diversity, and profiles of the following properties: molecular weight (MW); occurrence of rotatable bonds (RB); occurrence of hydrogen bond donors (HBD); and occurrence of hydrogen bond acceptors (HBA). It will be appreciated that in situations where there are more than two objectives, it is not possible to illustrate the trade-off between the objectives using simple 2D graphs. However, FIG. 10 illustrates a graph 1000 that is a parallel co-ordinates graph representation of the Pareto frontier shown in FIG. 5b. The horizontal axis represents two objectives, that is, molecular weight profile and diversity and the vertical axis represents the values of each objective. It will be appreciated that diversity is now represented as its complement, that is, (1-diversity) so that the direction of improvement in both objectives is towards zero on the y-axis. It will be appreciated that the two objectives have been standardised since they are plotted on the same scale. Each objective can be standardised independently by determining the maximum and minimum values for an objective. Each continuous line on the graph represents one solution in the current Pareto set. The competing nature of the objectives is shown by the intersections of the lines. It can be appreciated that an advantage of using parallel co-ordinates graphs to display a solution represented by a current Pareto set is that competition between different objectives is highlighted by the points of intersection.

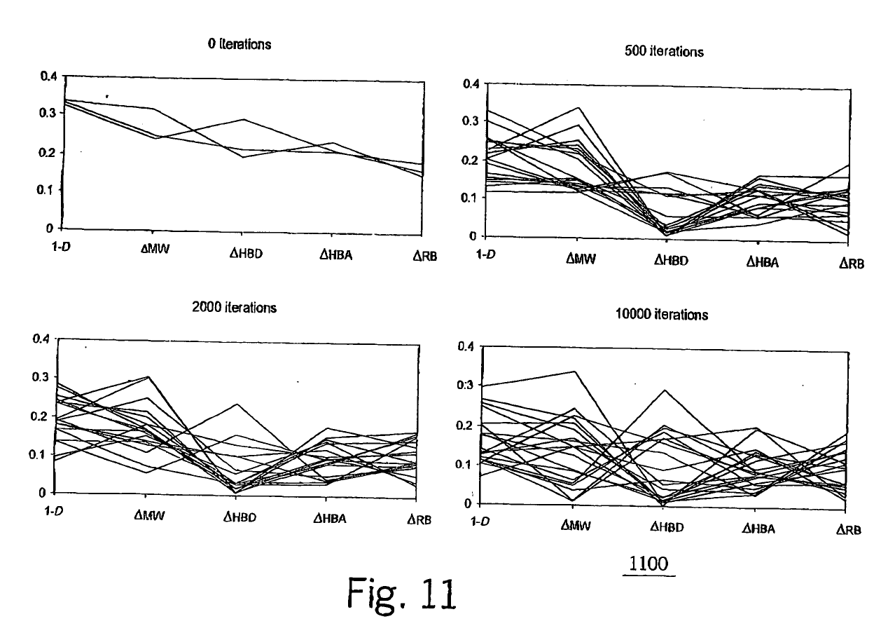

Referring to FIG. 11, there is shown a parallel coordinates graph representation 1100 of the multi-objective amide problem with snapshots taken at various stages of the search. The search was conducted for 5000 iterations. To compare the progress of the various objectives, all values have been standardised. Again, standardisation was achieved by determining maximum and minimum values for each objective. A value of zero represents the best value achievable when the objective is optimised alone. Furthermore, diversity is again represented as its complement, that is, (1-diversity), so that all objectives are minimised and the direction of improvement is the same for all objectives. The non-dominated solutions are shown in different stages of the search. It can be appreciated that as the search progresses, the solutions drift in the direction of multi-objective improvement, that is, the solutions tend towards lower values on the vertical axis. It can also be seen that as the search progresses the number of non-dominated solutions increases. Some competition is evident for example between HBA and HBD as is shown by the crossing lines in the graph. It can be appreciated that the relationships between pairs of objectives could be examined by re-ordering the objectives on the horizontal axis. Where there is no competition between objectives, that is, improvement in one corresponds to improvement in another, it is not necessary to include both objectives within the search process.

### EXAMPLE 6

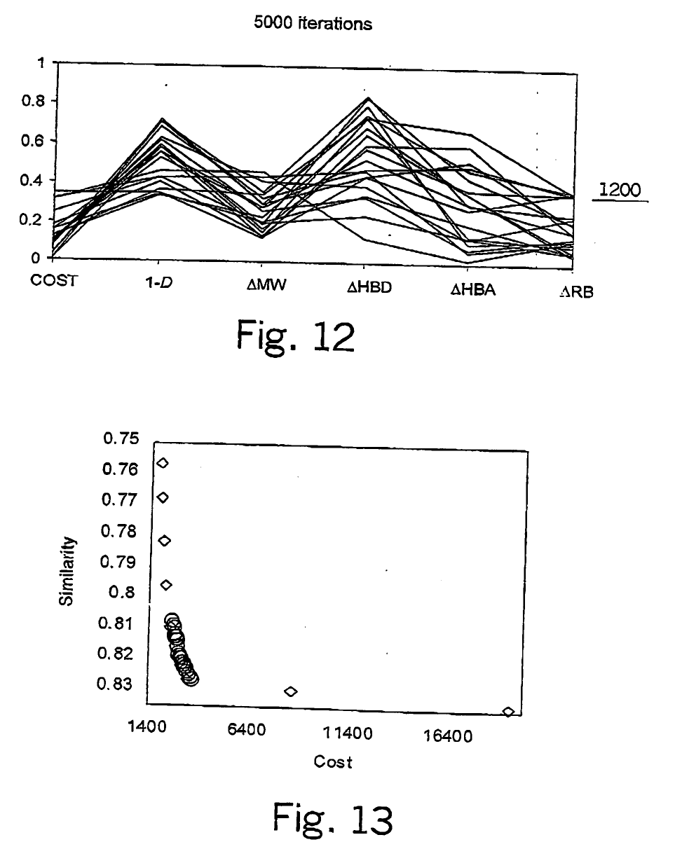

It will be appreciated that cost is an objective that should preferably be considered in the design of any combinatorial library. Referring to FIG. 12, there is shown the 2-aminothiazole library having been used to investigate the effect of including reactant cost as an objective in the search. The cost for each of the reactants was supplied. An embodiment of the present invention was configured to select 15×30 combinatorial subsets. The parallel co-ordinates graph 1200 shown in FIG. 12 shows the results of running an embodiment of the present invention using multiple objectives. In this embodiment, the distance-based diversity measure was replaced by a cell-based measure such as disclosed in “Partition-based selection. Perspect Drug Disc Design” Mason J S, Pickett S D, 1997: 7/8: 85-14 which is incorporated herein by reference for all purposes. Each product molecule in the virtual 2-aminothiazole library was assigned to a cell in a 3D space. The aim of this embodiment was to select 15×30 combinatorial subsets that occupy as many cells as possible within the 3D space, that have minimum cost and that have drug-like profiles of molecular weight, hydrogen bond donors, hydrogen bond acceptors and rotatable bonds.

### EXAMPLE 7

An embodiment of the present invention was configured to select 15×30 focused combinatorial subsets. Subset libraries were focused around a target compound by maximising the sum of normalised similarities of the compounds in the subsets to the target while simultaneously minimising the cost of the libraries. The parallel co-ordinates graph 1300 of FIG. 13 shows the results of running an embodiment of the present invention using multiple objectives of similarity to the target and cost.

Although the above embodiment has been described with reference to a method, the present invention is not limited thereto. Embodiments of the present invention can be implemented on a suitably programmed general purpose computer or in specifically designed computers/hardware. In particular, this invention may be used to program an automated chemical synthesis platform, such as the Advanced Chemtech 384. The design software would output a set of reagents which have been chosen to best meet the objectives set. In the most facile implementation, this would be a text file on a network computer disk, containing the names of the reagents and other relevant data, which could be read by the control software supplied with the synthesis platform. The control software would then enable an automated synthesis of the required library. There are other, more complex, methods by which this information could be transmitted. For example, the information could be transmitted through databases such as Microsoft Access or Oracle, or through scheduling software. However, in order to retain flexibility over the type of synthesis platform used, a text file is a preferred mechanism.

Although the above embodiments search for and present a Pareto optimal set of combinatorial libraries, the present invention is not limited to such an arrangement. Embodiments can be realised in which a Pareto set that is sub-optimal in some way may be selected. Alternatively, or additionally, embodiments can be realised in which a set of combinatorial libraries, other than a Pareto set, is selected from the recently updated population of combinatorial libraries.

Still further, although the above embodiments have been described with respect to the design of combinatorial libraries, the embodiments of the present invention are not limited thereto. Embodiments can be realised in which libraries other than combinatorial libraries are designed. For example, a near combinatorial library may be designed in which all combinations of the starting reagents do not appear in the final library, even though at least some combinations are included in the final library. Libraries other than combinatorial and near combinatorial libraries may also be designed using embodiments of the present invention.

1. A method for designing a set of libraries using a population of libraries, the method comprising performing, at least once, the steps of:
selecting at least a plurality of the libraries from the population of libraries;
applying genetic operators to selected, ranked, libraries to produce modified libraries;
calculating each of a plurality of objectives for each of the modified libraries;
calculating an associated dominance indication of each of the modified libraries;
ranking the modified libraries according to associated dominance indications;
incorporating the modified libraries into the population of libraries; and
forming the set libraries comprising selecting at least one library from the population of libraries.

2. A method as claimed in claim 1, in which the set of libraries is at least one of a set of combinatorial libraries or near combinatorial libraries.

3. A method as claimed in any preceding claim, in which the population of libraries is a population of combinatorial libraries or near combinatorial libraries.

4. A method as claimed in any preceding claim, in which the modified libraries are at least one of modified combinatorial libraries or modified near combinatorial libraries.

5. A method as claimed in any preceding claim, in which the step of selecting at least one library from the population of libraries comprises the step of selecting at least one combinatorial and/or near combinatorial library from the population of libraries.

6. A method as claimed in any preceding claim, in which the step of forming the set of libraries comprises the step of forming a Pareto set of libraries.

7. A method as claimed in claim 2, in which the Pareto set is a Pareto optimal set.

8. A method as claimed in any preceding claim, in which the plurality of objectives are specified via at least an n-dimensional vector function (f) of a population library (x) and at least two n-dimensional objective vectors (u=f(xᵤ) and v=f(xᵥ)).

9. A method as claimed in any preceding claim, in which the step of ranking the modified libraries comprises the step of determining an order of preference of the modified libraries.

10. A method as claimed in claim 9, in which the step of determining an order of preference of the modified libraries comprises determining that at least one of the objective vectors (u=[u₁, . . . , uₚ]) for a first modified library is preferable to the at least one of the objective vectors (v=[v₁, . . . , vₚ]) for a second modified library given a preference vector (g=[g₁, . . . , gₚ])
$$( {u ≺ v}_{g} )$$
if and only if
p=1=>(uₚ′ₚ<vₚ′)=>{(uₚ′=vₚ′)
\hat{ }[(vₚ*not≦gₚ*)=>(uₚ*ₚ<vₚ*)]}
and
p>1=>(uₚ′ₚ<vₚ′)=>{(uₚ′=vₚ′)
where u_{i, . . . ,p-1}=[uᵢ, . . . ,uₚ₋₁] and similarly for v and g; where the first kᵢ components of vectors uᵢ,vᵢ, and gᵢ are represented as uᵢ*, vᵢ*, and gᵢ*, respectively; the last nᵢ-kᵢ component of the same vectors are denoted uᵢ′, vᵢ′, and gᵢ′, also respectively; and the * and ′ indicate the components in which u either does or does not meet the goals.

11. A method as claimed in any preceding claim, in which the step of calculating the associated dominance indication of each of the modified libraries comprises determining whether at least a first objective vector (u=(u₁, . . . , uₙ)) for a first modified library has Pareto dominance over a second objective vector (v=(v₁, . . . , vₙ)) for a second modified library if and only if the u is partially less than v (uₚ<v) such that ∀iε{1, . . . ,n},uᵢ≦vᵢ\hat{ }∃iε{1, . . . ,n}:uᵢ<vᵢ.

12. A method as claimed in any preceding claim, in which the step of ranking the modified library comprises the steps of evaluating the preference of each modified library and ranking the modified library according to respective preferences.

13. A method as claimed in any preceding claim, in which the step of forming the set of libraries comprises the step of selecting the ranked modified libraries that are Pareto-optimal where a first library (xᵤ) of the population for a first objective vector is said to be Pareto-optimal if and only if there is no other library of the population for a second objective vector (xᵥ) for which the second objective vector, v=f(xᵥ)=(v₁, . . . , vₙ) dominates the first objective vector u=f(xᵤ)=(u₁, . . . , uₙ).

14. A method substantially as described herein with reference to and/or as illustrated in the accompanying drawings.

15. A system for designing a set of libraries using a population of libraries, the system comprising means for invoking, at least once,
means for selecting at least a plurality of the libraries from the population of libraries;
means for applying genetic operators to selected, ranked, libraries to produce modified libraries;
means for calculating each of a plurality of objectives for each of the modified libraries;
means for calculating an associated dominance indication of each of the modified libraries;
means for ranking the modified libraries according to associated dominance indications;
means for incorporating the modified libraries into the population of libraries; and
means for forming the set libraries comprising selecting at least one library from the population of libraries.

16. A system as claimed in claim 15, in which the set of libraries is at least one of a set of combinatorial libraries or near combinatorial libraries.

17. A system as claimed in any of claims 15 to 16, in which the population of libraries is a population of combinatorial libraries or near combinatorial libraries.

18. A system as claimed in any of claims 15 to 17, in which the modified libraries are at least one of modified combinatorial libraries or modified near combinatorial libraries.

19. A system as claimed in any of claims 15 to 18, in which the means for selecting at least one library from the population of libraries comprises means for selecting at least one combinatorial and/or near combinatorial library from the population of libraries.

20. A system as claimed in any preceding claim, in which the means for forming the set of libraries comprises means for forming a Pareto set of libraries.

21. A system as claimed in claim 20, in which the Pareto set is a Pareto optimal set.

22. A system as claimed in any of claims 15 to 21, in which the plurality of objectives are specified via at least an n-dimensional vector function (f) of a population library (x) and at least two n-dimensional objective vectors (u=f(xₙ) and v=f(xᵥ)).

23. A system as claimed in any of claims 15 to 22, in which the means for ranking the modified libraries comprises means for determining an order of preference of the modified libraries.

24. A system as claimed in claim 23, in which the means for determining an order of preference of the modified libraries comprises means for determining that at least one of the objective vectors (u=[u₁, . . . , uₚ]) for a first modified library is preferable to the at least one of the objective vectors (v=[v₁, . . . , vₚ]) for a second modified library given a preference vector (g=[g₁, . . . , gₚ])
$$( u {≺}_{g} v )$$
if and only if
p=1=>(uₚ′ₚ<vₚ′)=>55 (uₚ′=vₚ′)
\hat{ }[(vₚ*not≦gₚ*)=>(uₚ*ₚ<vₚ*)]}
and
p>1=>(uₚ′ₚ<vₚ′)=>{(uₚ′=vₚ′)
where u_{i, . . . ,p-1}=[uᵢ, . . . ,uₚ₋₁]and similarly for v and g; where the first kᵢ components of vectors uᵢ,vᵢ, and gᵢ are represented as uᵢ*, vᵢ*, and gᵢ*, respectively; the last nᵢ-kᵢ component of the same vectors are denoted uᵢ′, vᵢ′, and gᵢ′, also respectively; and the * and ′ indicate the components in which u either does or does not meet the goals.

25. A system as claimed in any of claims 15 to 24, in which the means for calculating the associated dominance indication of each of the modified libraries comprises means for determining whether at least a first objective vector (u=(u₁, . . . , uₙ)) for a first modified library has Pareto dominance over a second objective vector (v=(v₁, . . . , vₙ)) for a second modified library if and only if the u is partially less than v (uₚ<v) such that ∀iε{1, . . . , n},uᵢ≦vᵢ\hat{ }†iε{1, . . . ,n}: uᵢ<vᵢ.

26. A system as claimed in any of claims 15 to 25, in which the means for ranking the modified library comprises means for evaluating the preference of each modified library and ranking the modified library according to respective preferences.

27. A system as claimed in any of claims 15 to 26, in which the means for forming the set of libraries comprises means for selecting the ranked modified libraries that are Pareto-optimal where a first library (xᵤ) of the population for a first objective vector is said to be Pareto-optimal if and only if there is no other library of the population for a second objective vector (xᵥ) for which the second objective vector, v=f(xᵥ)=(v₁, . . . , vₙ) dominates the first objective vector u=f (xᵤ)=(u₁, . . . , uₙ).

28. A system substantially as described herein with reference to and/or as illustrated in the accompanying drawings.

29. A library design computer program element for implementing a method or system as claimed in any preceding claim.

30. A computer program product comprising a computer readable storage medium having stored thereon a computer program element as claimed in claim 29.

31. A method of manufacturing a library or element thereof comprising the steps of designing the library or element using a method, system, computer program element or computer program product as claimed in any preceding claim; and materially producing the designed library or element thereof.
